(19) United States
(12) Patent Application Publication
(10) Pub. No.: US 2012/0077849 A1
(43) Pub. Date: Mar. 29, 2012

(54) INHALER FOR DELIVERING A METERED DOSE

(75) Inventors: Sophie Eleanor D. Howson, Cambridgeshire (GB); Adrian James David Howson, Cambridgeshire (GB)

(73) Assignee: PRAGMATIC INNOVATION LIMITED, Huntingdon, Cambridgeshire (GB)

(21) Appl. No.: 13/263,331

(22) PCT Filed: Apr. 6, 2010
(86) PCT No.: PCT/GB10/50590

§ 371 Date: Dec. 15, 2011

(30) Foreign Application Priority Data
Apr. 6, 2009 (GB) 0905840.5

(51) Int. Cl. A61M 15/00; A61M 11/00; A61M 16/10; A61M 11/04; A61K 31/465; A61P 11/00

(52) U.S. Cl. 514/343; 128/203.12; 128/200.23; 128/203.15; 128/200.14; 128/203.21

(57) ABSTRACT

An inhaler for delivering a metered dose of a medicament, the inhaler comprising a dispensing port (15) and one or more containers (1, 2) holding a combination of a first drug and a second drug; wherein the first drug is for treatment of a physiological condition, and the second drug is a motivational substance; the inhaler including an actuator and a metered dispenser (3) for delivery when actuated of a predetermined dose of the first and second drugs to a patient by inhalation through the dispensing port (5).

### FIELD OF THE INVENTION

This invention relates to a dosage form for administration of drugs by inhalation, particularly but not exclusively to dosage forms for administration to habitual smokers.

This invention also relates to means for assisting patient compliance when using apparatus and methods for delivering a medicament using these dosage forms.

### BACKGROUND TO THE INVENTION

Chronic Obstructive Pulmonary Disease (COPD) is one of the largest causes of death worldwide. The economic burden of COPD is high, both financially and in terms of lost productivity. A primary risk factor for COPD is chronic tobacco smoking, and the vast majority of cases arise from this.

It is a well known problem that patient compliance (also called adherence) in all uses of inhalation therapies is very poor. Compliance is the ability of the patient to achieve the prescribed dose by means of inhalation i.e. bronchial administration. This may be due to poor technique or a lack of understanding of how to operate the inhaler, even when it is breath actuated.

Lack of compliance leads to poor quality of life for the patient and extra expense for the health provider when the patient spends longer than necessary in hospital.

Patient beliefs about COPD, as well as their motivation and expectations about the likelihood of success of medical interventions, can influence adherence rates. Other critical factors include the patient's understanding of their illness and therapy, and the complexity of the prescribed treatment regimen. Incorrect inhaler technique is also a common failing.

Smoking-related COPD patients are typically depressed by their condition and the prognosis. This causes many patients to seek the comfort of a cigarette, rather than give up smoking. Persistent smoking negates the benefits that the medicament can provide so that the patient becomes convinced that he or she is beyond hope.

Breath actuated inhalers, such as described in U.S. Pat. No. 7,219,664, where the delivered dose is only triggered when the patient breathes in correctly, spacers where the drug is generated as a cloud in a chamber which the patient breathes in) and education have only partially improved patient compliance.

Inhalers only act to control the delivery of drug if the patient is able to draw correctly on the inhaler. If the patient is not motivated to use their inhaler, additional features such as breath actuation do not make a difference.

Smoker COPD patients may have spent a lifetime inhaling a drug by means of a simple drug delivery device to achieve the maximum possible effect. The drug in this case is nicotine and the device is the cigarette.

Observation suggests that a patient who is a smoker has no problem drawing deeply on a cigarette but when presented with an inhaler is typically incapable of achieving the required function. Often, the patient will fail to take the inhaler with them—or they will forget to use it, or simply they will fail to see the medical benefits (perhaps because they continue to smoke) so they see no reason to continue using it.

Preferred embodiments of this invention use the patient's ability to draw on a cigarette and detect the effect of the motivational substance to improve inhaler technique.

### SUMMARY OF THE INVENTION

According to the present invention there is provided an inhaler for delivering a metered dose of a medicament;
the inhaler comprising a dispensing port and one or more containers holding a combination of a first drug and a second drug;
wherein the first drug is for treatment of a physiological condition, and the second drug is a motivational substance; the inhaler including an actuator and a metered dispenser for delivery when actuated of a predetermined dose of the first and second drugs to a patient by inhalation through the dispensing port.

In a preferred embodiment the first drug is for treatment of a pulmonary condition.

The second drug is preferably nicotine. Alternatively the second drug may be selected from the group consisting of:
nicotine mimicking substances, menthol, peppermint flavouring, other flavourings or analgesics. A preferred nicotine mimicking substance is lobeline.

The inhaler of this invention may be selected from the group consisting of:
a pressurised metered dose inhaler, a dry powder inhaler and a nebuliser.

The first drug may be selected from the group consisting of:
anaesthetics, anti-asthmatics, bronchodilators, anti-hypertensives, and antimicrobials, pulmonary surfactants and insulin. Tiotroprum and salbutamol are preferred first drugs.

Primarily this applies to smoking related Chronic Obstructive Pulmonary Disease but the motivational approach may also apply to asthma patients using a suitable motivational substance such as peppermint.

The invention finds particular application for patients where the application of the motivational substance will not re-kindle a former addiction to cigarettes. In the first instance, and for smoking-related COPD patients, the use of the invention may be restricted to recently diagnosed COPD patients or to persistent smoking COPD patients

Care may be needed to prevent the patient overdosing on prescribed nicotine. If each inhaled dose is limited to 1 mg, the total prescription may be limited to 40 doses within a safety limit.

Patient Controlled Analgesia is a procedure whereby the patient can operate a syringe pump via a button to deliver a dose of pain relief. After a prescribed number of doses in a defined time, the syringe pump does not deliver more analgesic but the patient still experiences a beneficial sensation consequent to the operation of the button.

In apparatus according to the present invention after a predetermined number of doses in a day, the motivational drug content, and the first drug may be reduced or be omitted. This feature may be implemented in a number of ways:

A capsule-based inhaler such as the Boehringer Ingleheim Handihaler may be prescribed with a daily series of capsules, the drug and motivational substance in each being reduced according to efficacy and safety limits.

By this means, the patient may experience a mitigation of craving within perhaps 5 daily doses, even though they may take more doses, some of which are in fact empty of motivational substance. On this basis, the patient may be prescribed a week's worth of medicament. Alternatively by exploitation of the drug half life, the patient may be prescribed more.

Lobelia (Lobelia inflata), also known as Indian tobacco, contains a substance (lobeline) that has some effects on the nervous system that are similar to the effects of nicotine. Pure lobeline or lobelia herb extract may be used to support smoking cessation. It may therefore represent an alternative motivational substance in place of nicotine. Other motivational substances include menthol and peppermint flavours and also some analgesics such as codeine.

An inhaler for delivering a metered dose of a medicament, the inhaler comprising a dispensing port and one or more containers holding a combination of a first drug and a second drug;
wherein the first drug is for treatment of a physiological condition, and the second drug is a motivational substance;
the inhaler including an actuator and a metered dispenser for delivery when actuated of a predetermined dose of the first and second drugs to a patient by inhalation through the dispensing port.

Thus the inhaler may comprise a pressurised metered-dose inhaler, in which case the motivational substance and second drug are stored together in a pressurised container or in separate pressurised containers. The first drug may be provided in a conventional metered dose inhaler canister or as a conventional dry powder dosage form. The inhaler may be a dry powder inhaler, or a nebulizer, in which case the first and second drugs are provided in the form of an aqueous mist. Alternatively the first and second drugs may be delivered as a powder mixture or as two separate powders, to be released from separate blisters (or other containers).

The form in which the motivational substance is stored in the container will depend upon the type of inhaler, but the motivational substance is suitable for providing to a user in the form of an aerosol. Examples of nicotine dosage forms are described in, for example, US 2008/302375, WO2008/140372 and EP1,663,168A. The nicotine may, be in the form of a coated product or absorbed onto fibres.

In an alternative embodiment, the inhaler may be configured such that a user is able to draw air through a mouthpiece, this air flowing past the container such that the first drug and motivational substance are entrained within the air flow.

In preferred embodiments the inhaler further comprises a system or device to automatically inhibit use after delivering one or more doses of the second drug. This may comprise, an electromechanical timer to inhibit the user from actuating the inhaler. Alternatively the device may be electronically controlled by the user, and may include a control system arranged to be electronically de-activated to prevent the user overdosing on drug when attempting to ingest more nicotine or other motivational substance. Alternatively, the patient may be prescribed a daily series of capsules for use in a capsule-based inhaler such as Handihaler® where the volumes of first drug and motivational substance are varied according to clinically-defined efficacy and safety needs.

The invention also provides a container for use with an inhaler as described above, containing one or a predetermined number of doses of a combination of nicotine or other motivational substance and a first drug.

In embodiments of this invention the inhaler and/or container is configured to vary the quantity of motivational substance delivered for example by providing a variable, in particular decreasing dose reduce a dependency on nicotine and remove a craving for cigarettes. After a course of treatment using the inhaler described herein, a patient may be better trained and more motivated to adhere to conventional treatments.

### DETAILED DESCRIPTION OF PREFERRED EMBODIMENTS

The invention is further described by means of example, but not in any limitative sense with reference to the accompanying drawings, of which;

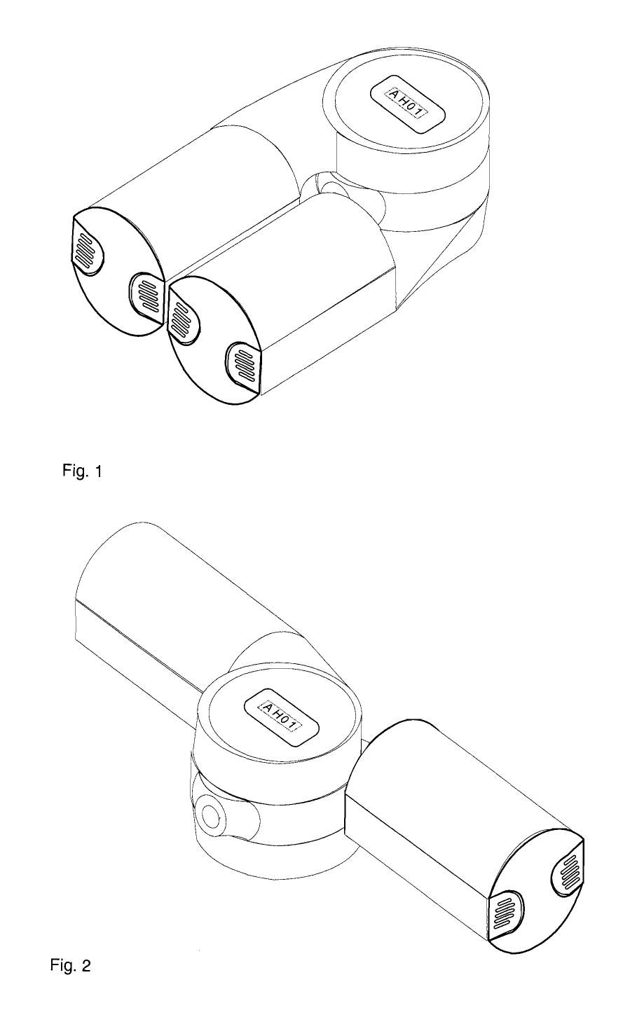

FIG. 1 shows a two chamber inhaler in accordance with this invention in the stowed position; and

FIG. 2 shows the inhaler of FIG. 1 in the deployed position.

FIG. 1 illustrates an inhaler in accordance with the present invention. Two drug containing containers (1, 2) are connected to a metered dose dispensing unit (3) having a visual display (4) and a dispensing port (5).

FIG. 2 shows the inhaler in the deployed position wherein the canisters (1,2) are rotated on rotatable mountings (6,7) to unlock and actuate an on-off switch on the dispenser (3). The display (4) may provide an indication of the number of available doses. Alternatively or in addition the number of doses which a patient is permitted to use may be provided.

Use of nicotine or similar substance to aid patient compliance when using inhalers in the population of smoking-related COPD (Chronic Obstructive Pulmonary Disease) is described.

A beneficial way to achieve compliance is to give the patient some means of feedback and motivation, when an inhaler has been correctly used.

In a cigarette, the feedback is the ‘hit’ that the smoker receives when the correct ingestion has been achieved. In the COPD patient, adding nicotine (or less addictive substance) to the existing formulation should therefore give the patient the same ‘hit’ when the inhalation has been correctly carried out. As in the case of learning to smoke, the patient will also optimise their technique to achieve the best result thereafter and will be motivated to keep using the device.

Combining of nicotine with other drugs, for example (and their brand names) include salbutamol (Ventolin), formoterol (Foradil, Oxis), salmeterol (Serevent) and terbutaline (Bricanyl) etc. can allow the particle distribution profile to be optimised for drug bioavailability at the same airflow rate and duration.

A typical dose of nicotine might be 1 mg per inhalation, equivalent to one cigarette or an equivalent dose of a less addictive substance. A typical dose of a drug in a dry powder inhaler can be up to 3 mg, the majority of which is a lactose excipient.

The use of nicotine will not work well for a non ex-smoker COPD patient nor for asthma patients, both of whom could be made ill by the effect of nicotine.

These patients or other users of inhalers may however benefit from a similar technique such as adding flavours (e.g. menthol) or aromas (e.g. lavender oil) to the inhaler to make it more attractive to the user to use, based on which compliance may be improved. Other examples may include combining the inhaler with a breathe-freshener product (eg peppermint). This is differentiated from other innovations whereby the drug is flavour-masked to make it more palatable.

For all of these users, the inhaler may be fitted with a lockout mechanism for example connected to the display to prevent excessive use—of the nicotine or of the therapeutic drug. Or, if the approach is implemented in a capsule-based inhaler, the volume of drug in the capsules may be titrated according to a defined pattern. Typically, inhaler drugs can be titrated to four doses per day. After four doses, the drug in the capsule or automatic device may not be delivered. After perhaps five doses the nicotine or less addictive substance may cease. Thereafter, the capsule may only contain excipient so that the patient only gains benefit from the placebo effect, an effect which is exploited in Patient Controlled Analgesics (PCAs).

The drug and nicotine or less addictive substance may be controlled by a valve within an automatic 2-channel device or, for example, by a designated set of capsules on a card indicating to the patient which ones to take first.

By this means, the patient may also be weaned off an addiction to nicotine by successive reduction in the nicotine delivered by the capsules or through the automatic device.

It is believed that once the patient has become more adept at using this form of inhaler with its motivational approach, they will be more likely to use conventional inhalers when subsequently prescribed.

Many other effective alternatives will occur to the skilled person. It will be understood that the invention is not limited to the described embodiments and encompasses modifications apparent to those skilled in the art lying within the scope of the claims appended hereto.

1-17. (canceled)

18. An inhaler for delivering a metered dose of a medicament, the inhaler comprising a dispensing port and one or more containers holding a combination of a first drug and a second drug; wherein the first drug is for treatment of a physiological condition, and the second drug is a motivational substance; the inhaler including an actuator and a metered dispenser for delivery when actuated of a predetermined dose of the first and second drugs to a patient by inhalation through the dispensing port.

19. An inhaler as claimed in claim 18 wherein the first drug is for treatment of a pulmonary condition.

20. An inhaler as claimed in claim 18 wherein the second drug is nicotine.

21. An inhaler as claimed in claim 18 wherein the second drug is selected from the group consisting of nicotine mimicking substances, menthol, peppermint flavouring, other flavourings and analgesics.

22. An inhaler as claimed in claim 21 wherein the nicotine mimicking substance is lobeline.

23. An inhaler as claimed in claim 18 selected from the group consisting of: a pressurised metered dose inhaler, a dry powder inhaler (bulk dose, pre-metered and capsule) and a nebuliser.

24. An inhaler as claimed in claim 18 wherein the first drug is selected from the group consisting of: anaesthetics, anti-asthmatics, bronchodilators, anti-hypertensive, antimicrobials, pulmonary surfactants, and insulin.

25. An inhaler as claimed in claim 18 wherein the first drug is selected from the group consisting of: salbutamol, formoterol, salmeterol, terbutaline, and tiotropium.

26. An inhaler as claimed in claim 18 configured such that a user is able to breathe air through said dispensing port said air flowing through said container such that said first drug and second drug are entrained in said air flow.

27. An inhaler as claimed in claim 18 wherein said container is removeable.

28. An inhaler as claimed in claim 18 further comprising a two-channel system or device automatically to inhibit use of the said device after delivering one or more said doses of said first drug to prevent overdose on the drug or on the motivational substance.

29. An inhaler container, in particular for the inhaler of claim 18, the container storing a combination of nicotine or a nicotine-mimicking agent and a first drug.

30. An inhaler as claimed in claim 18 further comprising a system with separate containers or containers for the drug or the motivational substance.

31. An inhaler as claimed in claim 18 further comprising a system with a regimen of capsules or blisters containing the dose or and nicotine (or less addictive substance) in pre-metered amounts, to prevent the patient from overdosing on the drug or the nicotine in any set period (eg 24 hours)

32. An inhaler as claimed in claim 18 further comprising a system with a regimen of capsules or blisters containing the dose or and nicotine (or less addictive substance) in pre-metered and successively declining amounts, to wean the patient off nicotine addiction over the duration of a course of treatement

33. A vapouriser for delivering a medicament, the inhaler comprising a mouthpiece, means for vapourising the active ingredient or ingredients of a plant material, and a container for a second drug, such that, in use, said second drug is delivered with said active ingredient or ingredients of said nicotine or less addictive substance to the user of said vapouriser.

34. A method of delivering a drug to a patient suffering from a smoking-related COPD, the method comprising delivering said drug to said patient in combination with nicotine or a less addictive agent.
